(19) United States
(12) Patent Application Publication
(10) Pub. No.: US 2014/0116443 A1
(43) Pub. Date: May 1, 2014

(54) EXPIRATORY THERAPY DEVICE

(71) Applicant: GALEMED CORPORATION, I-Lan Hsien (TW)

(72) Inventor: Gary C. J. LEE, I-Lan Hsien (TW)

(73) Assignee: GALEMED CORPORATION, I-Lan Hsien (TW)

(21) Appl. No.: 14/052,860

(22) Filed: Oct. 14, 2013

(30) Foreign Application Priority Data
Oct. 26, 2012 (TW) 101219930

(51) Int. Cl. A61M 16/20; A61M 16/00; A61M 16/04; A61M 16/10

(52) U.S. Cl. CPC A61M 16/20; A61M 16/105; A61M 16/0057; A61M 16/0488
USPC 128/204.25

(57) ABSTRACT

An expiratory therapy device includes: a flow-adjusting member defining an upper chamber and a lower chamber and having an inner tube that defines an inner chamber and that is formed with wall openings; a driving member; an opening-controlling member including a cylindrical valve that is formed with wall apertures and that is driven by the driving member; and a flow rate-enhancing member including a venturi tube that is connected to the flow-adjusting member, that has an air entrainment port, and that is provided with a jet-forming member defining a venturi orifice. The flow rate-enhancing member generates the venturi effect, such that outside air is entrained into the venturi tube through the air entrainment port and flows into the upper chamber.

### CROSS-REFERENCE TO RELATED APPLICATIONS

This application claims priority of Taiwanese Application No. 101219930, filed on Oct. 16, 2012.

### BACKGROUND OF THE INVENTION

1. Field of the Invention

This invention relates to an expiratory therapy device, more particularly to an expiratory therapy device defining an upper chamber and including a flow-rate enhancing member for increasing a flow rate of a gas flow to be delivered to the upper chamber.

2. Description of the Related Art

U.S. Pat. No. 5,598,839 discloses a positive expiratory pressure device that includes an input body with an inlet port, a one-way valve coupled to the inlet port, an inner plate formed with an opening, a hub supported in the input body through the inner plate and defining a central opening, and a pressure controller for selecting one of different-sized openings in a face plate which is mounted in the input body. In operation, when a patient inhales, outside air is drawn into the input body through the inlet port, the one-way valve, and the central opening, whereby the inhaled air is freely drawn into the patient's lungs. When the patient exhales, the exhaled air is prevented from passing through the central opening and the inlet port to the outside, and is permitted to pass only through the opening in the inner plate and the selected one of the openings in the face plate that is brought into alignment with the opening in the inner plate. As such, by repeatedly alternating alignment and misalignment between the selected one of the openings in the face plate and the opening in the inner plate at constant intervals, positive pressure pulses are created in the central opening, which results in lungs' vibration.

U.S. Pat. No. 4,538,604 discloses a respiratory system that includes a gas source for supplying a mixture of air and oxygen, a conduit line, a pressure regulator connected to the gas source and the conduit line for controlling the flow rate and the pressure of the mixture of air and oxygen in the conduit line, a control valve connected to the conduit line, and a tubular manifold connected to the conduit line and adapted to be inserted into the mouth of a patient. The control valve is operable for periodically interrupting the flow of the mixture through the conduit line so as to produce high frequency pressure pulses of the flow for delivery to the patient. Although the respiratory system can produce the pressure pulses in the conduit line, it has a high consumption of air supplied from the gas source.

### SUMMARY OF THE INVENTION

Therefore, an object of the present invention is to provide an expiratory therapy device that can overcome the aforesaid drawback associated with the prior art.

According to this invention, there is provided an expiratory therapy device that comprises: a flow-adjusting member including an outer shell, an inlet conduit that is connected to the outer shell, an outlet conduit that is connected to the outer shell, an inner tube that is surrounded by the outer shell, and a middle flange that is disposed between and that interconnects the outer shell and the inner tube and that cooperates with the outer shell and the inner tube to define an upper chamber and a lower chamber thereamong, the inlet conduit being in fluid communication with the outlet conduit through the upper chamber, the outer shell having a top closed end that defines a top side of the upper chamber, the inner tube defining an inner chamber and being formed with a plurality of wall openings that are angularly spaced apart from one another, the upper chamber being in fluid communication with the inner chamber; a driving member; an opening-controlling member including a cylindrical valve, the cylindrical valve being formed with a plurality of wall apertures that are angularly spaced apart from one another, and being disposed in the lower chamber, one of the cylindrical valve and the inner tube being rotatably sleeved on the other of the cylindrical valve and the inner tube, the driving member driving rotation of the cylindrical valve relative to the inner tube, such that the inner chamber is permitted to fluidly communicate with the lower chamber through the wall openings and the wall apertures when the wall apertures are brought into alignment with the wall openings, respectively, and that the wall openings are blocked by the cylindrical valve so that the inner chamber is not permitted to fluidly communicate with the lower chamber when the wall apertures are not in alignment with the wall openings; and a flow rate-enhancing member including a venturi tube provided with a jet-forming member that defines a venturi orifice, the venturi tube having an air entrainment port and defining a tube inner space, the jet-forming member being adapted to be connected to a pressurized gas source, and extending into the tube inner space, the venturi tube being connected to the inlet conduit so as to permit delivery of a pressurized gas from the pressurized gas source into the upper chamber through the flow rate-enhancing member, the flow rate-enhancing member generating the venturi effect, such that outside air is entrained into the venturi tube through the air entrainment port to mix with the pressurized gas in the venturi tube when the pressurized gas passes through the venturi orifice into the tube inner space.

### BRIEF DESCRIPTION OF THE DRAWINGS

In drawings which illustrate an embodiment of the invention,

FIG. 1 is a schematic exploded view of the preferred embodiment of an expiratory therapy device according to the present invention;

FIG. 2 is an exploded perspective view of an assembly of a flow-adjusting member, a driving member and an opening-controlling member of the preferred embodiment;

FIG. 3 is an assembled perspective view of the assembly of the flow-adjusting member, the driving member and the opening-controlling member of the preferred embodiment; and

FIG. 4 is a sectional view of the assembly of the flow-adjusting member, the driving member and the opening-controlling member of the preferred embodiment.

### DETAILED DESCRIPTION OF THE PREFERRED EMBODIMENT

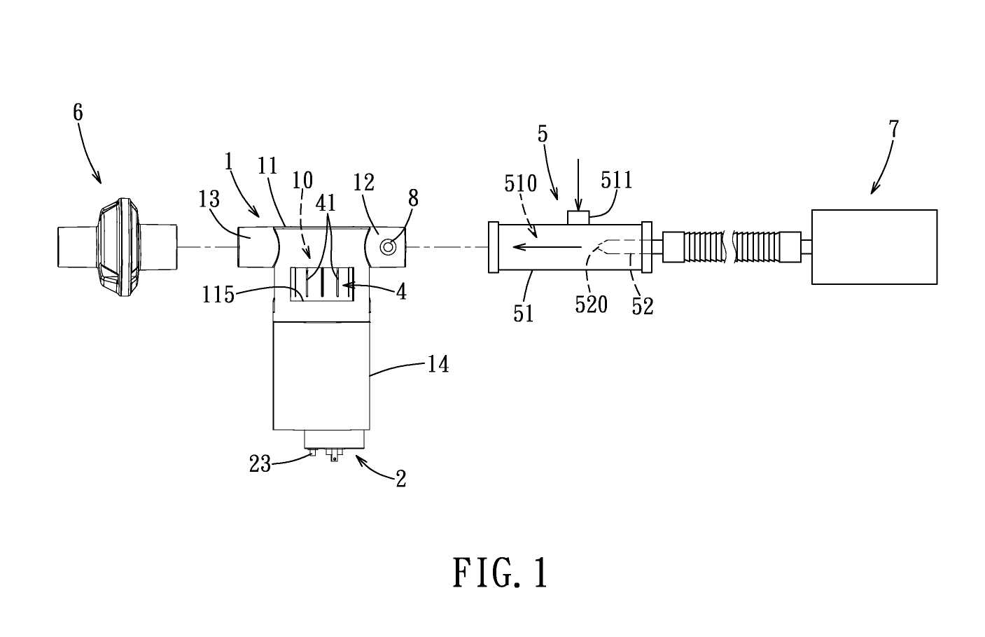

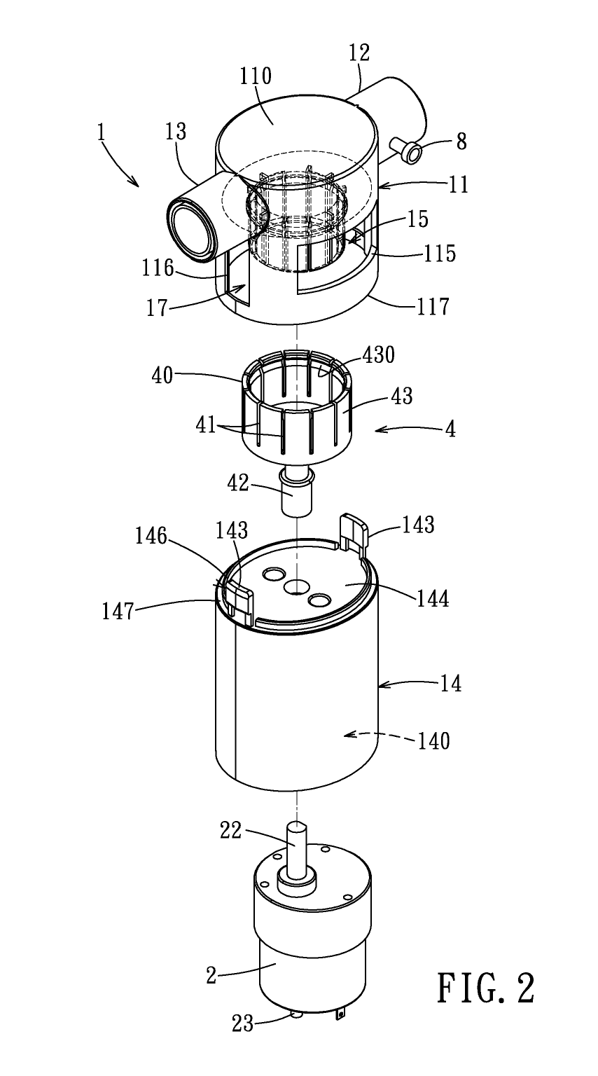

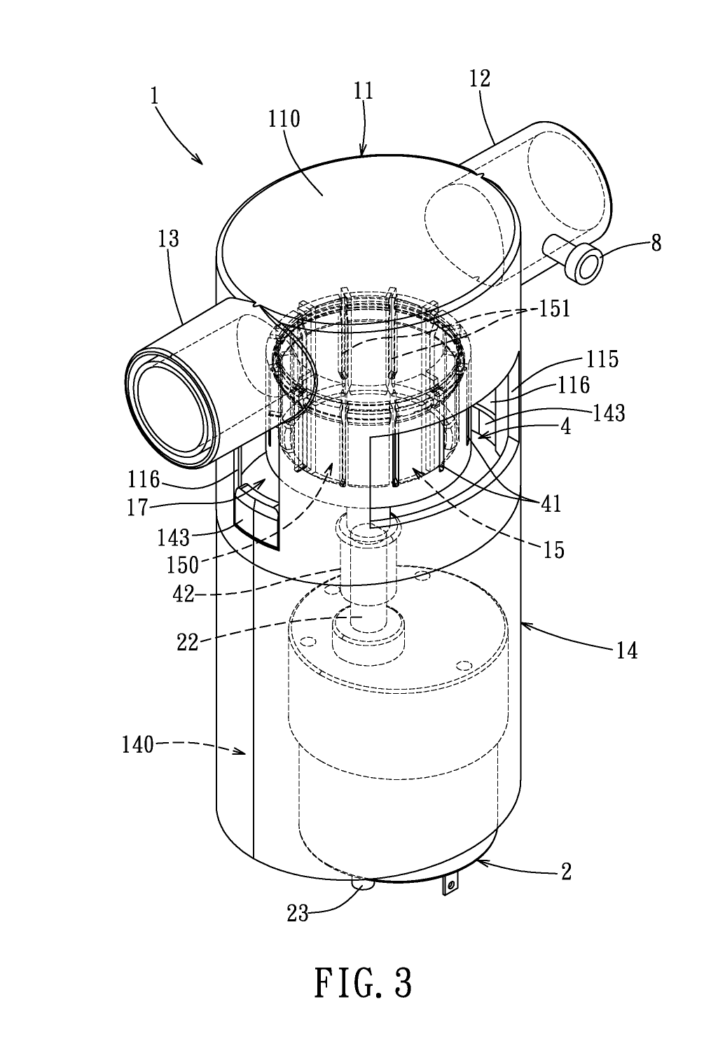

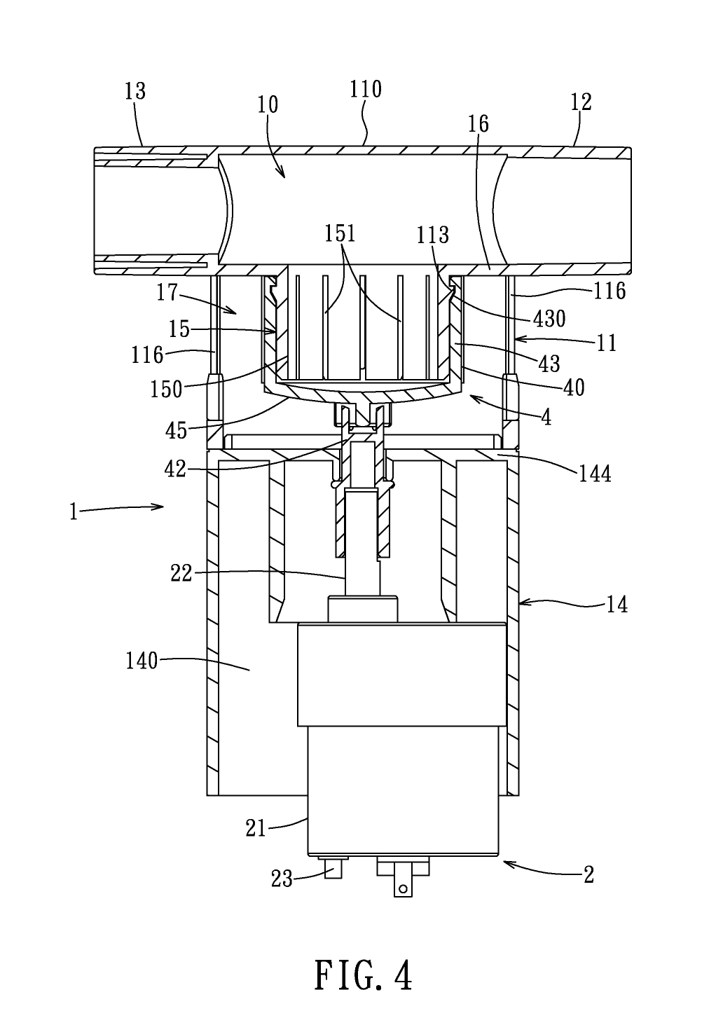

FIGS. 1 to 4 illustrate the preferred embodiment of an expiratory therapy device according to the present invention. The expiratory therapy device includes a flow-adjusting member 1, a driving member 2, an opening-controlling member 4, a flow rate-enhancing member 5, a cylindrical motor housing 14, a pressure relief valve 8, a filter 6, and a pressurized gas source 7.

The flow-adjusting member 1 includes an outer shell 11, an inlet conduit 12 that is connected to the outer shell 11, an outlet conduit 13 that is connected to the outer shell 11, an inner tube 15 that is surrounded by the outer shell 11, and a middle flange 16 that is disposed between and that interconnects the outer shell 11 and the inner tube 15 and that cooperates with the outer shell 11 and the inner tube 15 to define an upper chamber 10 and a lower chamber 17 thereamong. The inlet conduit 12 is in fluid communication with the outlet conduit 13 through the upper chamber 10. The pressure relief valve 8 is connected to the inlet conduit 12. The outer shell 11 has a top closed end 110 that defines a top side of the upper chamber 10. The inner tube 15 defines an inner chamber 150, and is formed with a plurality of wall openings 151 that are angularly spaced apart from one another. The upper chamber 10 is in fluid communication with the inner chamber 150. The filter 6 is connected to the outlet conduit 13, and is adapted to be connected to a mouth piece (not shown) for insertion into a patient's mouth.

The driving member 2 includes a motor 21, an output shaft 22, and a speed regulating knob 23 for adjusting a speed of the output shaft 22.

The opening-controlling member 4 includes a cylindrical valve 40 and a shaft coupler 42. The cylindrical valve 40 is formed with a plurality of wall apertures 41 that are angularly spaced apart from one another, is disposed in the lower chamber 17, and is rotatably sleeved on the inner tube 15. Alternatively, the inner tube 15 can be rotatably sleeved on the cylindrical valve 40 (not shown). The cylindrical valve 40 has a bottom wall 45 that covers a bottom of the inner tube 15, and a surrounding wall 43 that extends from the bottom wall 45, that is coaxially sleeved on the inner tube 15, and that is formed with the wall apertures 41. The shaft coupler 42 is disposed below and is connected to the bottom wall 45 of the cylindrical valve 40. The output shaft 22 interconnects the motor 21 and the shaft coupler 42. The driving member 2 drives rotation of the cylindrical valve 40 relative to the inner tube 15, such that the inner chamber 150 is permitted to fluidly communicate with the lower chamber 17 through the wall openings 151 and the wall apertures 41 when the wall apertures 41 are brought into alignment with the wall openings 151, respectively, and that the wall openings 151 are blocked by the surrounding wall 43 of the cylindrical valve 40 so that the inner chamber 150 is not permitted to fluidly communicate with the lower chamber 17 when the wall apertures 41 are not in alignment with the wall openings 151.

The flow rate-enhancing member 5 includes a venturi tube 51 provided with a tubular jet-forming member 52 that defines a venturi orifice 520. The venturi tube 51 has an air entrainment port 511 that is disposed adjacent to the venturi orifice 520, and defines a tube inner space 510. The jet-forming member 52 is connected to the pressurized gas source 7, and extends into the tube inner space 510. The venturi tube 51 is connected to the inlet conduit 12 so as to permit delivery of a pressurized gas, such as air or oxygen, from the pressurized gas source 7 into the upper chamber 10 through the flow rate-enhancing member 5. The flow rate-enhancing member 5 generates the venturi effect, such that outside air is entrained into the venturi tube 51 through the air entrainment port 511 to mix with the pressurized gas in the venturi tube 51 when the pressurized gas passes through the venturi orifice 520 into the tube inner space 510, thereby increasing a flow rate of a gas flow (i.e., the mixture of the entrained air and the pressurized gas) to be delivered from the venturi tube 51 into the upper chamber 10. As an example, the flow rate of the gas flow can be increased from 15 liters/minute (without the flow rate-enhancing member 5) to about 80 to 100 liters/minute (with the flow rate-enhancing member 5). Hence, a low pressure of the pressurized gas source can be used for the expiratory therapy device and consumption of the pressurized gas can be considerably reduced with the inclusion of the flow rate-enhancing member 5 in the expiratory therapy device. In addition, the higher the flow rate of the gas flow into the upper chamber 10, the higher will be the positive pressure built in the upper chamber 10, which is important for providing sufficient and effective therapy to the patient's lungs.

The cylindrical motor housing 14 defines an accommodating space 140, and has a top wall 144 and a top rim 146 which extends upwardly from and which cooperates with the top wall 144 to define an annular shoulder 147 therebetween. The top wall 144 defines a top side of the accommodating space 140. The driving member 2 is mounted in the accommodating space 140. The outer shell 11 further has a bottom open end 117 that is seated on the annular shoulder 147, and is formed with a vent window 115 and a pair of engaging holes 116. The motor housing 14 further has a pair of engaging ears 143 that extend upwardly from the annular shoulder 147 and that engage respectively the engaging holes 116 in a snap-fit engaging manner. The lower chamber 17 is in fluid communication with the outside of the outer shell 11 through the vent window 115. The shaft coupler 42 extends into the accommodating space 140.

The surrounding wall 43 of the cylindrical valve 40 is formed with an annular inner groove 430. The inner tube 15 is formed with an annular tongue 113 that engages movably the annular inner groove 430 in a tongue-and-groove engaging manner.

With the inclusion of the flow rate-enhancing member 5 in the expiratory therapy device of the present invention, the aforesaid drawback associated with the prior art can be alleviated.

While the present invention has been described in connection with what is considered the most practical and preferred embodiment, it is understood that this invention is not limited to the disclosed embodiment but is intended to cover various arrangements included within the spirit and scope of the broadest interpretation and equivalent arrangements.

What is claimed is:

1. An expiratory therapy device comprising:
a flow-adjusting member including an outer shell, an inlet conduit that is connected to said outer shell, an outlet conduit that is connected to said outer shell, an inner tube that is surrounded by said outer shell, and a middle flange that is disposed between and that interconnects said outer shell and said inner tube and that cooperates with said outer shell and said inner tube to define an upper chamber and a lower chamber thereamong, said inlet conduit being in fluid communication with said outlet conduit through said upper chamber, said outer shell having a top closed end that defines a top side of said upper chamber, said inner tube defining an inner chamber and being formed with a plurality of wall openings that are angularly spaced apart from one another, said upper chamber being in fluid communication with said inner chamber;
a driving member;
an opening-controlling member including a cylindrical valve, said cylindrical valve being formed with a plurality of wall apertures that are angularly spaced apart from one another, and being disposed in said lower chamber, one of said cylindrical valve and said inner tube being rotatably sleeved on the other of said cylindrical valve and said inner tube, said driving member driving rotation of said cylindrical valve relative to said inner tube, such that said inner chamber is permitted to fluidly communicate with said lower chamber through said wall openings and said wall apertures when said wall apertures are brought into alignment with said wall openings, respectively, and that said wall openings are blocked by said cylindrical valve so that said inner chamber is not permitted to fluidly communicate with said lower chamber when said wall apertures are not in alignment with said wall openings; and
a flow rate-enhancing member including a venturi tube provided with a jet-forming member that defines a venturi orifice, said venturi tube having an air entrainment port and defining a tube inner space, said jet-forming member being adapted to be connected to a pressurized gas source, and extending into said tube inner space, said venturi tube being connected to said inlet conduit so as to permit delivery of a pressurized gas from the pressurized gas source into said upper chamber through said flow rate-enhancing member, said flow rate-enhancing member generating the venturi effect, such that outside air is entrained into said venturi tube through said air entrainment port to mix with the pressurized gas in said venturi tube when the pressurized gas passes through said venturi orifice into said tube inner space.

2. The expiratory therapy device of claim 1, wherein said cylindrical valve has a bottom wall that covers a bottom of said inner tube, and a surrounding wall that extends from said bottom wall, that is coaxially sleeved on said inner tube, and that is formed with said wall apertures.

3. The expiratory therapy device of claim 2, wherein said driving member includes a motor and an output shaft, said opening-controlling member further including a shaft coupler that is disposed below and that is connected to said bottom wall of said cylindrical valve, said output shaft interconnecting said motor and said shaft coupler.

4. The expiratory therapy device of claim 3, further comprising a cylindrical motor housing that defines an accommodating space and that has a top wall and a top rim which extends upwardly from and which cooperates with said top wall to define an annular shoulder therebetween, said top wall defining a top side of said accommodating space, said motor being mounted in said accommodating space, said outer shell further having a bottom open end that is seated on said annular shoulder and being formed with a vent window and a pair of engaging holes, said motor housing further having a pair of engaging ears that extend upwardly from said annular shoulder and that engage respectively said engaging holes in a snap-fit engaging manner, said lower chamber being in fluid communication with the outside of said outer shell through said vent window, said shaft coupler extending into said accommodating space.

5. The expiratory therapy device of claim 2, wherein said surrounding wall of said cylindrical valve engages movably said inner tube in a tongue-and-groove engaging manner.
